(19) United States
(12) Patent Application Publication
(10) Pub. No.: US 2026/0091160 A1
(43) Pub. Date: Apr. 2, 2026

(54) SYNTHETIC HEMOCOMPATIBLE VASCULAR GRAFTS MANUFACTURED FROM POLYVINYL ALCOHOL-BASED BIOMATERIALS

(71) Applicant: Nedeljko Vladimira Gvozdic, Fort Myers, FL (US)

(72) Inventor: Nedeljko Vladimira Gvozdic, Fort Myers, FL (US)

(21) Appl. No.: 19/339,507

(22) Filed: Sep. 25, 2025

Related U.S. Application Data
(60) Provisional application No. 63/700,370, filed on Sep. 27, 2024.

(51) Int. Cl. A61L 27/50; A61L 27/16; A61L 27/34; A61L 27/58; B33Y 80/00; C08L 29/04

(52) U.S. Cl. CPC A61L 27/507; A61L 27/16; A61L 27/34; A61L 27/58; B33Y 80/00; C08L 29/04

(57) ABSTRACT

Synthetic hemocompatible vascular grafts manufactured from polyvinyl alcohol-based biomaterials containing crystalline segments of polyvinyl alcohol, which are long-term fully functioning vascular grafts that provide no activity towards immune systems in recognizing surfaces as foreign bodies that would normally lead to fibrosis, platelet activation and subsequent thrombi formation, and thus occlusion of the grafts.

### CROSS-REFERENCE TO RELATED APPLICATIONS

This application is a utility application claiming the benefit and priority of U.S. Provisional application Ser. No. 63/700,370, filed Sep. 27, 2024.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

There are numerous patents describing manufacturing synthetic vascular grafts and the like, for example, Gore Medical holds numerous patents related to GORE-TEX® vascular grafts made from expanded polytetrafluoroethylene such as U.S. Pat. No. 5,800,512 that describes a method for forming a multilayer vascular graft with enhanced porosity and strength; U.S. Pat. No. 6,719,783, that describes a microporous vascular graft with enhanced radial strength and suture hole elongation resistance; U.S. Pat. No. 6,436,135 that discloses a prosthetic vascular graft; U.S. Pat. No. 5,866,217 that discloses a silicone composite vascular graft designed for dialysis applications; U.S. Pat. No. 5,735,892 that discloses a tubular intraluminal graft with a stent and polytetrafluoroethylene coverings; U.S. Pat. No. 11,547,548 that discloses a vascular graft incorporating a stent; U.S. Pat. No. 12,193,957 that discloses a stent/graft with a constraining line; U.S. Pat. No. 5,910,168 that discloses a synthetic vascular graft that offers reduction in blood loss when the graft is punctured by suturing needles; U.S. Pat. No. 8,343,567 that discloses a method of treating the surface of a medical device with a biomolecule.

Also known is U.S Patent publication U.S. 2016/0361462 that discloses modifications of the biomaterial made from PVA with surface topographical features; U.S. patent publication U.S. 2012/0041539 that discloses protein iocoacervates and biomaterials graft system in making the grafts; U.S. 2014/0309726 that discloses biodegradable scaffolds for in-situ tissue engineering, and U.S. Pat. No. 10,603,156 that discloses a vascular graft that includes a biodegradable polyester electrospun tubular core made out of biodegradable polyester

This instant invention deals in one embodiment with novel polyvinyl alcohol hydrogels and solids and their use in providing physiological and mechanical biocompatible artificial vascular grafts.

“Biocompatibility” as used in this invention is used only as a general term because biocompatibility is not precisely defined in the field. Biocompatibility means different things for different applications such as cardiovascular, cosmetic surgery, ophthalmic work, and so on.

Thus, the inventor addresses distinct families of biocompatibility requirements for biomaterials, that is, “physiological” and “mechanical” biocompatibility. A better understanding of biocompatibility and a need for or precise requirements relates to biocompatibility that is driven by the development of non-immunogenic biomaterials of the present invention.

The biomaterials of the instant invention have a non-immunogenic response, that is, the family of biomaterials of the instant invention deprives the human immune system of having cellular and molecular mechanisms to identify surfaces of biomaterial of the present invention as foreign bodies and eliminates activation of molecular and cellular components of immune system at the site of direct contact and creating delayed activation that may cause unwanted immune activity downstream.

The overall objective of the instant invention is the development of a long-term solutions to making vascular grafts especially having internal diameters smaller than 6 mm without occlusion and activation of any components of the immune system in the blood stream.

There are different modes of operation and different uses of the family of polyvinyl alcohol hydrogels made using mixed solvents whose properties are fully customizable allowing practically an infinite number of polyvinyl alcohol hydrogels. Biomaterials are made having specific single or multiple gradient properties.

Surface properties of all kinds of polyvinyl alcohol based biomaterials can be customized using a wide range of parameters to arrive at surface properties that closely resemble native properties of human tissue as well as proving scaffolding that will support and drive tissue engineering as part of the regenerative medicine carried out in the desired direction of the present invention. This is accomplished by a combination of activities provided by such fully customizable scaffolding such as customizing physical/mechanical properties so that cells can adhere and receive the desired signaling based on hardness of the surface and strength of adhesion.

Surace modification provides desired levels of physiological biocompatibility so that surface of non-immunological biomaterials assume certain desired levels of immunological visibility to the cell and to the immune system so that adhesion and cell migration can take place, when necessary.

Surface modification can take place in any desired way to direct migration of the cells in a preferred direction by custom modification of the hardness and adhesion to provide the desired migration path for the cells. Use of controlled release capability of the hydrogel of the instant invention that can benefit from the gradient properties of the hydrogel which can direct the rate and direction of the release of growth factors, chemokines, cytokines, and other physiologically and thus cellularly active entities to precisely guide regenerative processes as well as re-establishing homeostasis for transplanted cells of transplanted entire tissue such as cells, in addition to the use of Extracellular Matrix (ECM) which is important in transplantation of individual and clusters of cells.

Thus, the vascular grafts of this invention can be made in various thicknesses, water content, levels of single or gradient levels of biostability with different rates of bioerosion, levels of biocompatibility that includes physiological and mechanical biocompatibility, tensile strength, modulus, a degree of reinforcement that is typically done using long fibers in the form of mats or woven and non-woven fibrous structures, the use of biostable fiber like polyvinyl alcohol and polyethylene, with modified surfaces, to form hydrogen bonds, the use of biodegradable fibers such as those made from biodegradable polymers including collagen and other similar options.

Polyvinyl alcohol fibers as well as silk, and fibers based on surface modified polymers such as polyethylene are used for reinforcement and sutures. Cyanoacrylate glues can be used to seal and to immobilize the vascular grafts, for example, to prevent blood leaks at the site of anastomosis and the like.

Thus, what is disclosed and claimed in the instant invention, in one embodiment, is a physically crosslinked polyvinyl alcohol hydrogel prepared using mixed solvents. The hydrogel contains continuous, uninterrupted immobilized water. The hydrogels have at least 88% by weight water content or higher and a syndiotacticity of fifty percent or greater.

The hydrogels of this invention are unique. They must have a syndiotacticity of fifty percent or greater, have a molecular weight of at least 550 and up to 200,000, preferable, a molecular weight distribution of 2.5 or lower, a head-to-tail monomer orientation of 98 percent or higher, total branching of 1 percent or lower, total disordered stereotacticity of 70 percent or lower and a preferred degree of hydrolysis of 99.95 percent or greater.

### BRIEF SUMMARY OF THE INVENTION

What is disclosed and claimed herein in one embodiment is a synthetic hemocompatible vascular graft manufactured from physically crosslinked polyvinyl alcohol-based biomaterials. The polyvinyl alcohol-based biomaterials have a water content of at least 88 weight percent, a molecular weight equal to or greater than 100,000 g/mole, a molecular weight distribution of 2 to 6 units, a degree of hydrolysis of 99.5% or greater, a syndiotacticity of fifty percent or greater, a head-to-tail monomer orientation of 98 percent or higher, total branching of 1 percent or lower, total disordered stereotacticity of 70 percent or lower, and the ability to form preferably nano-size crystalline domains that consistently remain under the submicron in size under specific processing conditions.

The vascular grafts have the properties such as biocompatibility, internal diameters of 1 mm or greater, resistance to mechanical fatigue, customizability, negative foreign body recognition while implanted in a human body, elasticity, easily reinforced, resistance to physiological system activation, biologically and physiologically stable, chemically stable, and accepts and holds sutures.

One can create crystalline sites in the PVA depending on what is desired in the final PVA product, which provides physical crosslinking and self-reinforcement. Lower number of crystallites means softer, more pliable materials having less strength than materials having significantly large numbers of crystallites, which can be correlated to weight percent of crystalline domains.

In another embodiment, there is a process for manufacturing a synthetic hemocompatible vascular graft. The process comprises providing a polyvinyl alcohol polymer having a molecular weight equal to or greater than 100,000 g/mole; mixing the polyvinyl alcohol polymer with a mixed solvent solution in a vessel; heating the mixture under a nitrogen blanket while stirring for at least 2 hours; removing all of said mixed solvent except traces of said solvent; forming said vascular graft; cooling said vascular graft at less than 0° C. for at least 2 hours to at least form some crystalline domains; allowing said vascular graft to come to room temperature; extracting the mixed solvent from the graft.

In still another embodiment there is a bio-erodible synthetic hemocompatible vascular graft manufactured from polyvinyl alcohol-based biomaterials, said polyvinyl alcohol-based biomaterials having a water content of at least 88 weight percent and a molecular weight of 5,000 g/mole to 80,000 g/mole.

Yet another embodiment of this invention is a synthetic hemocompatible vascular graft when coated. The coating is selected from an enzymatically stable non-bio-erodible polyvinyl alcohol hydrogel combined with enzymatically unstable bio-degradable biomaterial such as alginate, POLY (LACTIC ACID) HOMO-POLYMER, GLA, and PEG; or electrospun polyvinyl alcohol fibers combined with enzymatically unstable bio-degradable such biomaterial as alginate, PLA, GLA, and PEG; or a bio erodible cellular or non-cellular polyvinyl alcohol hydrogel.

The processing of the polyvinyl alcohol as set forth in this invention allows for a resulting microstructure that is uniform and consists of nano-size crystalline domains imbedded into a continuous amorphous matrix of polyvinyl hydrogel which allows for achieving maximum possible reinforcement of the hydrogel matrix as well as providing non-thrombogenic surfaces.

### BRIEF DESCRIPTION OF THE SEVERAL VIEWS OF THE DRAWING(S)

FIG. 1 is an example of a sub-millimeter vascular graft made from bulk bio erodible polyvinyl alcohol hydrogel.

FIG. 2 is shows three vascular grafts of different lengths with 2.0 mm inner diameter and 0.8 mm thick walls.

FIG. 3 is shows a vascular graft having an inner diameter of 3.0 mm and a wall thickness of 0.5 mm.

FIG. 4 shows a conical vascular graft having an inner diameter at one end of 7.0 mm and at the other end 3.0 mm. The wall thickness is 1.0 mm.

FIG. 5 shows a vascular graft with 3.5 mm inner diameter and 0.8 mm thick walls. It is reinforced with wound multi-filament braided silk sutures/fibers with 100 μm diameters.

FIG. 6 shows vascular grafts having a 5 mm (upper graft) internal diameter and 4 mm (lower graft) internal diameter.

FIG. 7 shows a vascular graft with a 4 mm inner diameter and 1 mm thick walls. Multifilament braided cellulose fibers with a diameter of 800 μm diameter reinforces the graft.

FIG. 8 shows a vascular graft with an internal diameter of 1.8 mm and a wall thickness of 0.5 mm.

FIG. 9 shows a vascular graft reinforced with cloth.

FIG. 10 shows a vascular graft with a 4.5 mm inner diameter and 1.0 mm thick wall reinforced with a gauze-like cloth.

FIG. 11 shows continuous hydrogel tubing with an internal diameter of 0.7 mm and a wall thickness of about 0.3 mm that can be cut to length for vascular grafts.

FIG. 12 shows a tubing with an internal opening of 0.5 mm and a wall thickness of about 0.3 mm that can be cut to length for vascular grafts.

FIG. 13 shows polyvinyl alcohol hydrogel tubing with an internal opening of 0.8 mm and 1.2 mm inner diameter with a wall thickness of about 0.3 to 0.5.

FIG. 14 shows hydrogel tubing with an inner opening of 0.7 mm and a wall thickness of about 0.3 mm made by extrusion.

FIG. 15 shows composite polyvinyl alcohol hydrogel vascular grafts useful in wall regeneration of vasculature in the body.

FIG. 16 shows silk fiber reinforcement for vascular grafts.

FIG. 17 shows vascular grafts using polyvinyl alcohol fibers for reinforcement.

FIG. 18 shows a vascular graft with a 3.5 mm inner diameter and 1.5 mm thick walls made from polyvinyl alcohol hydrogel reinforced with wound silk fibers.

FIG. 19 shows a vascular graft that has had its pores filled with Bioglass.

FIG. 20 shows a cloth reinforced vascular graft with a 4.0 inner diameter and 1.0 mm thick walls.

FIG. 21 shows a composite vascular grafts made only from PVA hydrogels.

FIG. 22 shows a vascular graft with embedded sutures for anastomoses of synthetic vascular graft of this invention.

FIG. 23 shows a vascular graft having embedded sutures.

FIG. 24 shows embedded silk sutures in a vascular graft.

FIG. 25 shows embedded carbon fibers as sutures for vascular grafts.

FIG. 26 shows embedded silk sutures that are slightly thicker than FIG. 24.

FIG. 27 shows a silk suture embedded into each end of the vascular graft.

### DETAILED DESCRIPTION OF THE INVENTION

Significant efforts have been made to develop a family of vascular graft-like devices using a PVA-based family of biomaterials, for example, working prototypes of “general” purpose vascular grafts, vascular access grafts and vein-grafts intended for vein valve replacement. Vascular grafts for microsurgery including capillaries, small diameter vascular grafts and vascular grafts with large bore, vascular access grafts such as AV grafts and vein valve grafts have been made and tested in vivo. A key prerequisite for successful development of all these devices is to have fully non-immunogenic/hemocompatible surfaces of the synthetic vascular graft lumen to provide non-thrombogenic responses in the presence of blood.

Recent advances in development of a family of PVA-based biomaterials of the present invention have further revealed additional aspects of the inherent nature of these biomaterials, which provide additional key benefits for design of a new generation of cardiovascular devices.

It is particularly important to emphasis that practically all properties of the new generation of PVA-based biomaterials can be fully customized to closely mimic vascular tissues they will replace. The new family of these biomaterials allows for the design of a simple method for anastomosis that is particularly important for microvascular surgeries. Extensive testing of mechanical properties and hemocompatibility of various prototypes of vascular devices indicates that polyvinyl alcohol hydrogels of the instant invention meet all prerequisites for successful long-term operation of practically all devices in direct contact with blood.

The new generation of PVA-based biomaterials not only satisfy the key requirements to maintain surfaces non-thrombogenic, but they also go a step further, as they deprive the innate immune system from activating molecular or cellular mechanisms to recognize these surfaces as foreign bodies or takes away an ability to recognize its surfaces potentially as “self”, or even recognize their existence. This is because cells cannot “probe” the nature of the surfaces of these hydrogels since cells cannot establish cell traction force (CTF), a prerequisite for the ability of cells to probe the nature and recognize the existence of any surfaces.

An important requirement for devices made for cardiovascular application is that the materials must endure hundreds of millions of bendings, that is, compressions and/or extensions without material fatigue. Our extensive mechanical testing for loss of mechanical strength due to fatigue indicates that 20 million bending's cause no detectable loss in mechanical properties. This means that vein valves that undergo approximately 1 million bendings a year can last for at least 20 years.

However, there are strong indications that biomaterials of the present invention are likely to meet a much larger number of bending cycles without loss of mechanical properties. These test data indicate that this biomaterial, possessing a maximum level of plasticization practically possible, will provide the maximum level of molecular mobility to accommodate for external molecular stresses and thus make fatigue an unimportant phenomenon in the use of these biomaterials in cardiovascular applications.

An initial objective of the present invention is the development of permanent replacements of missing portions of blood vessels using “classical” permanently implantable hemocompatible biomaterial. The long term objective, defined in the embodiments of the present invention, is to take advantage of unique set of properties of the new generation of PVA Hydrogels to regenerate in situ native vascular tissue and eliminate a need for the use of permanent, implantable synthetic vascular grafts as well as a new generation of non-drug eluting stents.

This can accomplished by using biomaterials of the present invention that are fully customizable, which allows the design of composite bio-erodible scaffolding that possesses necessary temporal and spatial distribution of gradient properties that are necessary for regeneration of a complex cardiovascular tissue.

With respect to the property of biocompatibility, the specific PVA hydrogels of this invention are highly biocompatible.

With respect to the property of internal diameter of 1 mm or greater please see examples

With respect to the wall thickness of the grafts they range from 200 to 1,500 μm which insures integrity.

With respect to resistance to mechanical fatigue properties, the instant invention can control the water content of the PVA hydrogels to control tensile strength, tensile modulus, and pore sizes.

With respect to the customizability property of the devices the instant invention allows for the formation of bifurcated grafts, for example side arms to the vascular grafts. This is accomplished in one method using 3-D printing to manufacture the graft and yet, another method is molding the entire bifurcated graft at one time. Still another is to attach another tube, that is, another vascular graft by cutting a hole into the wall of one graft tube and gluing the side arm graft to it using self-adhesion of the polyvinyl hydrogels or using cyanoacrylate adhesives.

With respect to the elasticity property, devices of this invention are soft and gentle to adjacent tissue and are elastomeric and pliable to accommodate or adopt to the shape of the space of any site in which they are used.

With respect to sutures in the vascular grafts, see example 4 infra.

The vascular grafts of this invention made from PVA hydrogels can be reinforced with fibers, especially braided fiber, woven, and non-woven fibers. This allows for the walls of the grafts to be orders of magnitude stronger and improves burst strength, wherein proper braiding prevents kink formation of the grafts.

The grafts of this invention can be customized, and they can allow surface modification that will provide a desired level of physiological biocompatibility so surfaces of non-immunological biomaterial assume a certain desired level of immunological “visibility” to the tissue cells and to the immune system so that cell adhesion, division, differentiation, and cell migration can take place during tissue regeneration.

Surface modification to these biomaterials can take place in any desired manner to direct migration of cells in the preferred direction and at a rate that is preferred by custom modification of hardness and adhesion based on chemical modification of surfaces to guide the migration path of cells thus controlling how tissue regeneration occurs.

Using the controlled release capability of the hydrogel is critical for regenerative processes to take place at all and be controlled by desired kinetics. Controlled release capability is augmented by gradient properties of the hydrogel which can direct the rate and direction of the release growth factors, chemokines, cytokines, and other physiologically and thus cellularly active entities to precisely guide the regeneration process as well as re-establish homeostatsis of regenerated tissue or transplanted cells or transplanted entire tissue such as cells plus ECM.

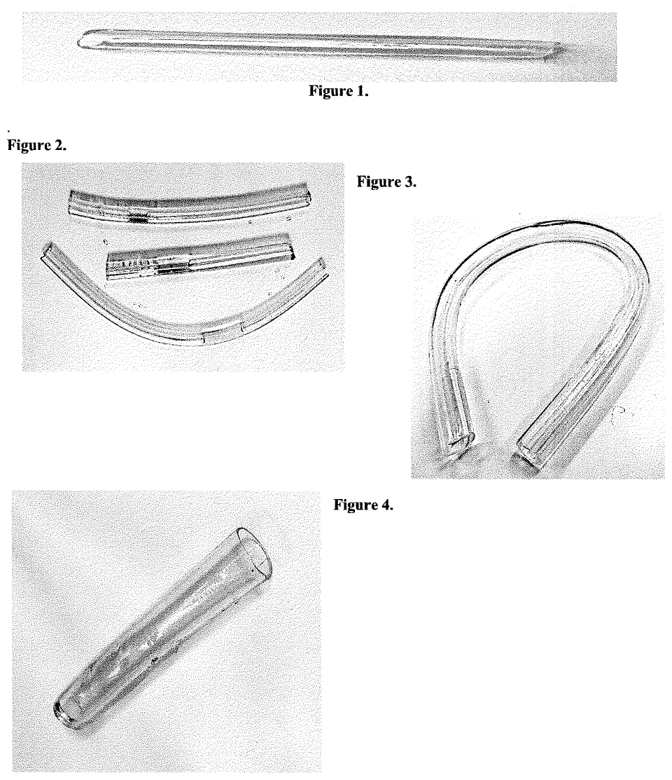

FIG. 1 is an example of a sub-millimeter vascular graft made from bulk bio erodible polyvinyl alcohol hydrogel. FIG. 2 shows three vascular grafts of different lengths with 2.0 mm inner diameter and 0.8 mm thick walls. The tube of the vascular graft was made from bulk PVA polymer which had a molecular weight of about 150,000 and greater than 99.95% of hydrolysis and was made using extrusion. No fibers or textile reinforcement was used.

FIG. 3 is shows a vascular graft having an inner diameter of 3.0 mm and a wall thickness of 0.5 mm. FIG. 4 shows a conical vascular graft having an inner diameter at one end of 7.0 mm and at the other end 3.0 mm. The wall thickness is 1.0 mm. This graft was made by molding without any textile or other reinforcement.

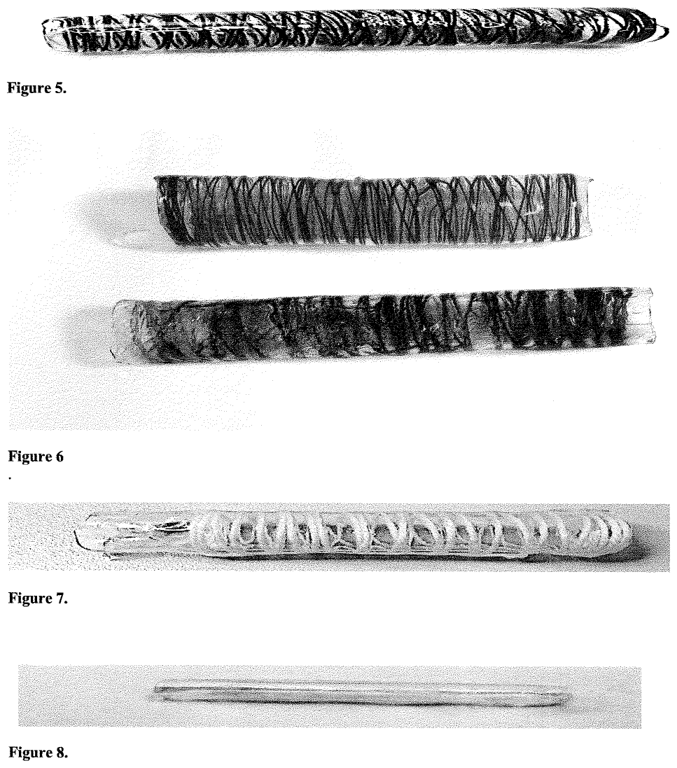

FIG. 5 shows a vascular graft with 3.5 mm inner diameter and 0.8 mm thick walls. It is wound with multi-filament braided silk sutures/fiber with 100 μm diameters. The fibers are cohesively bonded to the polyvinyl alcohol hydrogel of the present invention because they can form H-bonds with the hydrogel. FIG. 6 shows vascular grafts having a 5 mm (upper graft) internal diameter and 4 mm (lower graft) internal diameter using the same format as the grafts in FIG. 5.

FIG. 7 shows a vascular graft with a 4 mm inner diameter and 1 mm thick walls. Multifilament braided cellulose fibers with a diameter of 800 μm diameter reinforces the graft. The graft was prepared manually by winding braided cellulose fibers around a glass rod with a 4 mm diameter and then the rod was submerged into a solution of the PVA.

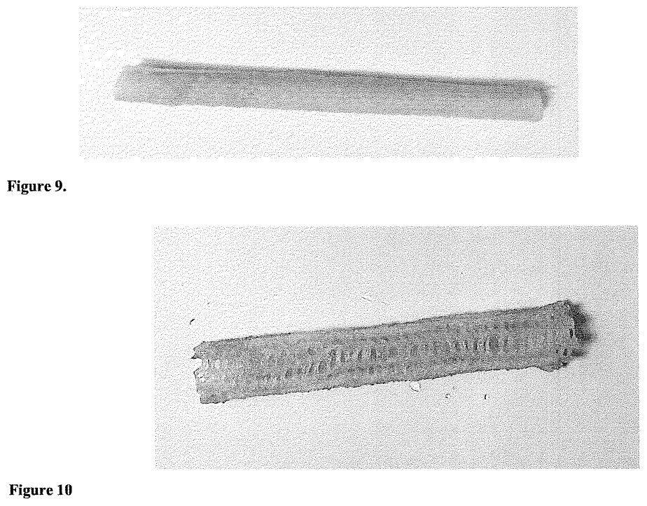

FIG. 8 shows a vascular graft with an internal diameter of 1.8 mm and a wall thickness of 0.5 mm. This graft was prepared as in the graft in FIG. 7 and a tubular mold was used to shape the outside surfaces. FIG. 9 shows a vascular graft reinforced with cloth. The graft was prepared by wetting both surfaces of single layer of textile with PVA by submersing the textile sheet into a PVA solution. Once the textile sheet had been completely wetted, it was wound around a glass rod with a 3.0 mm. diameter and then submerged again into the solution. The tubular mold was used again to shape the material.

FIG. 10 shows a vascular graft with a 4.5 mm inner diameter and 1.0 mm thick wall reinforced with a gauze-like cloth.

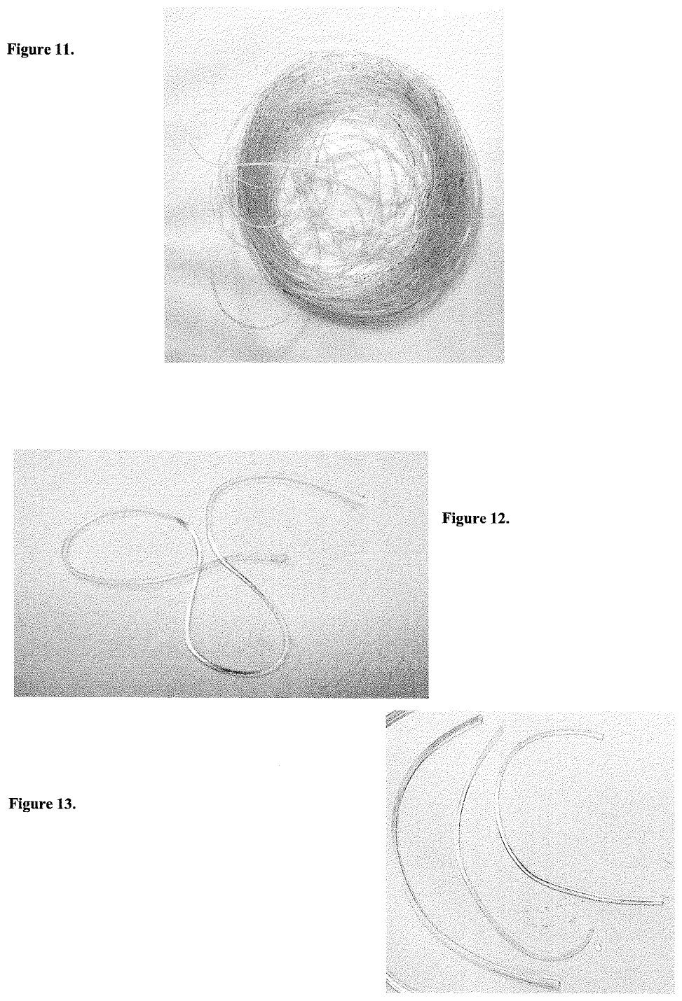

FIG. 11 shows continuous hydrogel tubing with an internal diameter of 0.7 mm and a wall thickness of about 0.3 mm that can be cut to length for vascular grafts. The tubing was extruded on an extruding device that would produce tens to hundreds of meters of tubing from this hydrogel. Various lengths of grafts were cut, sterilized, packaged, and used in animal testing using microsurgical vascular techniques. Some of the grafts were sutured to the wall of natural microvascular blood vessels using standard anastomosis.

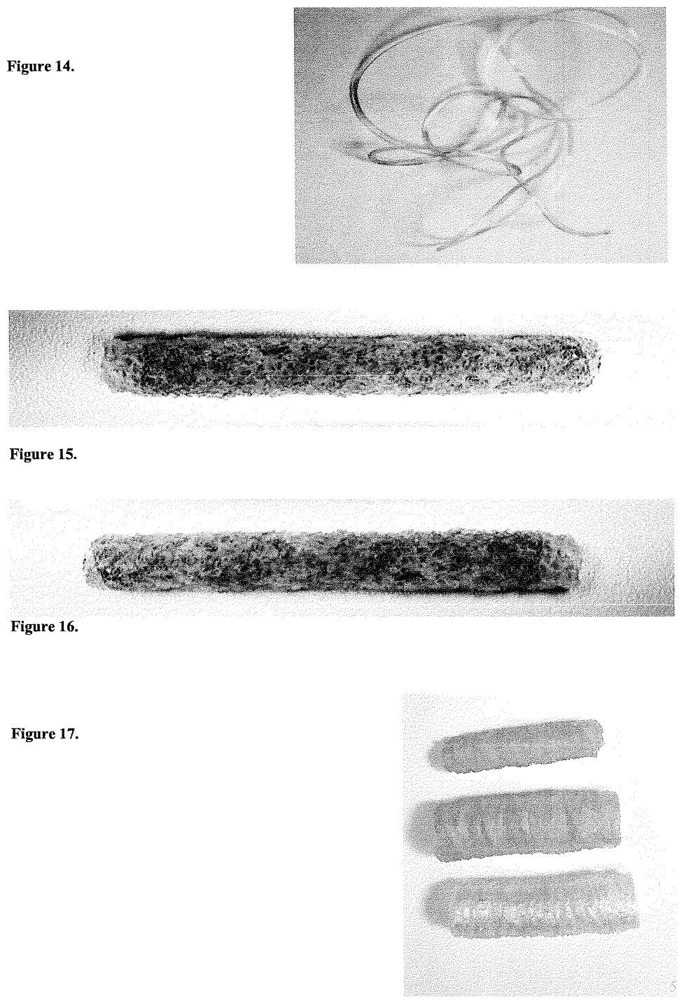

FIG. 12 shows a tubing with an internal opening of 0.5 mm and a wall thickness of about 0.3 mm that can be cut to length for vascular grafts. FIG. 13 shows polyvinyl alcohol hydrogel tubing with an internal opening of 0.8 mm and 1.2 mm inner diameter with a wall thickness of about 0.3 to 0.5. FIG. 14 shows hydrogel tubing with an inner opening of 0.7 mm and a wall thickness of about 0.3 mm made by extrusion. FIG. 15 shows composite polyvinyl alcohol hydrogel vascular grafts useful in wall regeneration of vasculature in the body. The enhancement comes from embedding Bioglass to induce tissue regeneration.

FIG. 16 shows showing silk fiber reinforcement.

FIG. 17 shows vascular grafts using polyvinyl alcohol fibers for reinforcement.

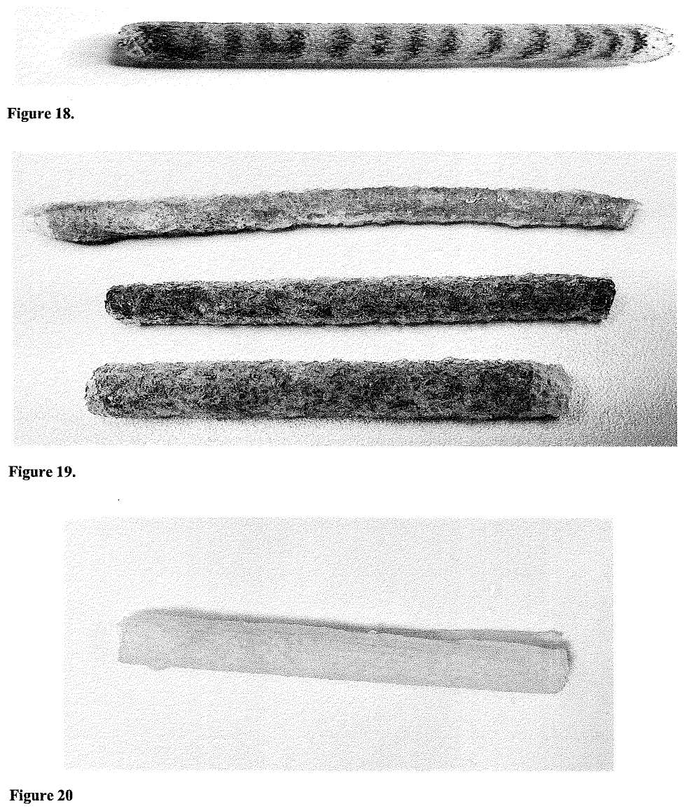

FIG. 18 shows a vascular graft with a 3.5 mm inner diameter and 1.5 mm thick walls made from composite PVA hydrogels. The inner layer was made from bulk hydrogel that provides a smooth, non-thrombogenic surface for blood flow. The outer layer is cellular PVA made using a pore forming method using pre-selected sizes and shapes of salt particles that were then removed by washing out the salt. The cellular wall was reinforced with multifilament silk fibers.

FIG. 19 shows a vascular graft that has had its pores filled with Bioglass.

FIG. 20 shows a cloth reinforced vascular graft with a 4.0 inner diameter and 1.0 mm thick walls.

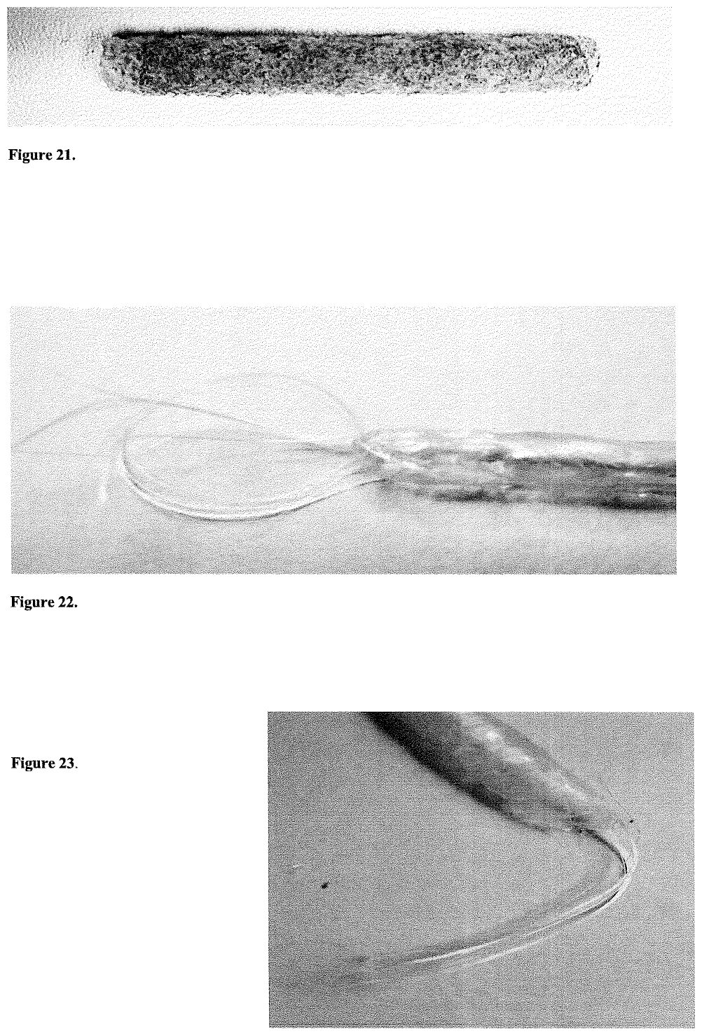

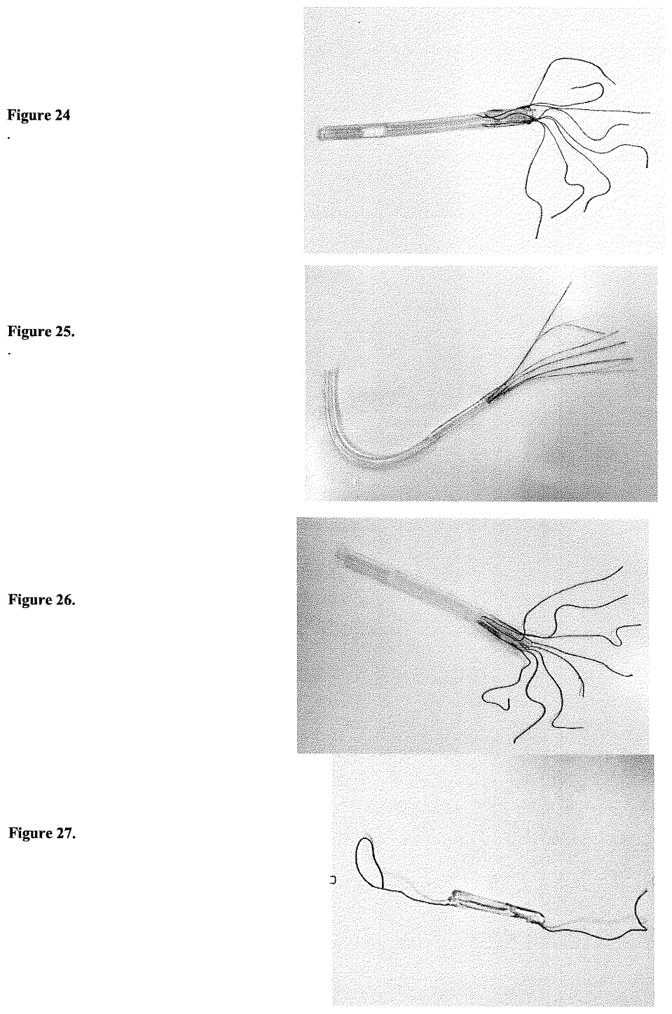

FIG. 21 shows a composite vascular grafts made only from PVA hydrogels. FIG. 22 shows a synthetic vascular graft with embedded multiple sutures of this invention. FIG. 23 shows a vascular graft having embedded sutures. FIG. 24 shows silk sutures in a vascular graft. FIG. 25 shows embedded carbon fibers as sutures. FIG. 26 shows silk sutures that are thicker than FIG. 24. FIG. 27 shows a silk suture embedded into each end of the vascular graft.

### EXAMPLES

When used herein, “low viscosity” means 200 cps to 1,000,000 cps at 25°. When used herein, “low temperature” means −35° C. to 40° C.

### Example 1. Example of Biomaterial Preparation for Vascular Grafts

This example deals with making enzymatically stable PVA Hydrogels having mechanical and biocompatibility properties with particular emphasis on long-term hemocompatibility suitable for making vascular grafts. Hydrogels of this invention are made using mixed solvents based on dimethyl sulfoxide (DMSO) and other solvents.

A poly (vinyl alcohol) polymer having a 150,000 molecular weight, a viscosity of 67 cps, in a 4 weight percent aqueous solution, and having 99.95% degree of hydrolysis was dissolved in a mixed solvent composed of 70/30 weight percent dimethyl sulfoxide (DMSO) and water. A PVA solution having a concentration of 10% wt./wt. of PVA, was made by placing 20 gms of PVA in a powdery form into a dissolution kettle containing 180 gms of the mixed solvent.

Dissolution was carried out at 90° C. in a water bath under a nitrogen atmosphere blanket over the PVA solution while continuously stirring for four hours. This resulted in a low viscosity PVA solution, which was used for making PVA hydrogel bulk and cellular hydrogel tubing by molding, extrusion, casting and 3-D Printing.

Objects made from this PVA solution were subjected to cooling in a refrigerator/freezer at a temperature of −18° C. for 8 hours. It is critical to emphasize that only one cooling cycle is required. No Freeze and Thaw cycling is permitted in this invention since multiple Freeze and Thaw cycles will result in hazy hydrogels and loss of control of kinetics of crystallite formation. The cooling process initiates the formation of submicron crystallites that act as crosslinkers and reinforcing agents resulting in physically crosslinked fully transparent hydrogels.

When DMSO was used as co-solvent for making physically crosslinked PVA hydrogels, the DMSO needs to be removed from hydrogel objects used in certain medical applications because it may cause lysis of certain cells especially of red blood cells. DMSO is removed from these hydrogels by a common extraction process such as either by placing these hydrogels into a water bath for 3 hours, then replacing water in the bath four times every 3 hours or using continuous extraction such as in Soxhlet extractor for complete removal of DMSO. Yet in another example, running water was used for extraction of DMSO from thin walls of hydrogel tubes by using 0.5 liter of water/minute flow rate into a submersion bath for a duration of 90 minutes.

This process of preparation of physically crosslinked PVA Hydrogel tubes for vascular grafts results in hydrogels that closely mimic mechanical properties of the walls of actual vasculature. These hydrogels can be used either non-reinforced, that is, “as is” bulk hydrogel or reinforced by continuous fibers, braided fibers, woven and non-woven fibers. Wall thicknesses of hydrogel tubes intended for vascular grafts ranged typically from 200 to 1,500 μm.

### Example 2. Example of Biomaterial Preparation for Vascular Grafts and Vascular Graft Tissue Bioengineering

This example deals with making enzymatically stable PVA Hydrogels having mechanical and biocompatibility properties with particular emphasis on long-term hemocompatibility suitable for making vascular grafts. PVA hydrogels in this example are made using mixed solvents based on glycerin and water.

Glycerin is chosen as a co-solvent since glycerin does not have a strong lysis action on cell membranes. Hence, when making these PVA hydrogels, glycerin does not need to be completely removed as in the case of Example 1. Other hydrophilic solvents that do not interfere with lysis of cells and hemocompatibility can also be used to make PVA solutions. This example follows the same procedure as outlined in the Example 1 except the following:

The mixed solvent for dissolution of PVA powder was made using glycerin instead of DMSO. The mixed solvent in this example has the following composition: 8% glycerol and 92% water. The same dissolution and physical crosslinking procedures were used here as described in the Example 1. The composition of mixed solvent in this Example is used in those cases when PVA solutions are used for vascular graft tissue printing, that is, when one needs to incorporate living cells into the PVA hydrogel matrix where the PVA matrix serves not only as a hemocompatible wall of tubing intended for vascular graft but also as an extracellular matrix (ECM) for imbedding cells for vascular graft bioengineering.

### Example 3. Procedure for Making Enzymatically Stable Vascular Grafts Using Bulk and Cellular PVA Hydrogels

This example deals with making hemocompatible enzymatically stable bulk PVA Hydrogels for embedding cells required for vascular tissue engineering using manufacturing processes such as 3-D tissue printing, tissue extrusion, tissue molding and use of any other known manufacturing procedure that can provide embedding of desired cells withing the wall of the vascular graft without inducing destruction to the imbedded cells caused by the solvent, temperature or shear, or other forces generated during the manufacturing tissue engineering construction. In this example, PVA hydrogels are made from mixed solvents based on glycerin or other hydrophilic solvents that are “cell membrane friendly”, to prevent cell lysis or induce cell apoptosis.

The same procedure as outlined in Example 2 is used here except the following: once the glycerin/water solution of PVA has been made by dissolution of PVA powder by heating to about 90° C., the temperature of the PVA solution is lowered to about 40° C. (typically temperatures of solution were between 3° and 45° C.). Desired cells were added into the low viscosity PVA solution in glycerin-based mixed solvent to generate 68 population of cells within the PVA solution and gently mixed to uniformly disperse the cells. During the addition of the cells and gentle mixing, the container was blanketed with an atmosphere enriched with oxygen.

This PVA solution can be used for manufacturing vascular grafts using 3-D tissue printing, tissue extrusion, tissue molding or similar processing that does not cause destruction of imbedded cells in the PVA hydrogel matrix. The continuous PVA hydrogel matrix serves as vascular walls as well as an extracellular matrix (ECM) for the cells.

It is important to emphasize that these tissue manufacturing processes based on PVA solutions made by glycerin as a key component in the mixed solvent are used at temperatures 30° C. to 45° C. or as low as about 10° C. to minimize temperature caused stress to the cell population and also to slow the metabolism of the cells to reduce loss of cell population during manufacturing. Vascular devices, housing cells imbedded in the bulk hydrogel, were made in the form of a tube.

### Example 4. Vascular Grafts with Imbedded Sutures

This example deals with a method of imbedding sutures into walls of a vascular graft. The importance of imbedded sutures is in doing an anastomosis of a vascular graft of any bore size but particularly of a small diameter vascular graft. Since all non-reinforced hydrogels have low tear strength, imbedded sutures in this invention provide benefits over classical suturing because they provide tear-free suturing of hydrogels. This is because one does not need to puncture a wall of the hydrogel vascular graft which eliminates generation of tear initiation sites in the wall of vascular graft because sutures are already imbedded in the hydrogel. This suturing creates leak free anastomosis, and rapid and simple suturing.

One can use classical suturing by penetrating the ends of vascular graft with a suture and tying the suture to the wall of a blood vessel. There are multiple problems with the use of classical suturing of hydrogels. For instance, when the suture penetrates through the hydrogel it forms the hole, which will be enlarged when the suture is under tension, and the suturing will come loose. Also, such an enlarged hole in the hydrogel easily undergoes tears because all hydrogels are well known to have a low tear strength. Also, enlarged holes are the site of blood leaks at the site of anastomosis.

A more desirable method is a method of suturing at the site of anastomosis using sutures imbedded into hydrogel walls which completely eliminate drawbacks of classical suturing. The criterion for the selection of material for sutures that can be imbedded into PVA hydrogels is that the material from which sutures are made must be capable of forming hydrogen bonds with the PVA hydrogel matrix and thus it must be cohesively bonded (not adhesively bonded) to the PVA hydrogel. This means that the suture will never be detached or pulled out of the hydrogel, or de-bonded, unless the hydrogel matrix undergoes tearing and thus full mechanical destruction.

Materials that can be used for making such sutures are PVA fibers, silk, cellulose-based fibers, or any other material such as surface-modified PE fibers that are capable of forming hydrogen bonds with the PVA hydrogel matrix. PVA fibers are preferred because of their intrinsic high strength that is higher than any other known man-made fiber, especially if the PVA fibers are ultra-drawn.

Suture imbedding was done manually into wall of vascular grafts at the stage prior to subjecting vascular graft construct to low temperature induced hydrogel crosslinking, though robotic arm with precision placement of sutures into the wall of vascular graft is preferred.

### Example 5. Vascular Grafts with Scaffolding on the Surface

This example deals with methods of making a vascular graft that has on its outer surface a scaffolding made from different biomaterials that will guide and accelerate integration of a vascular graft with the surrounding tissue. This example deals with the construction of scaffolding on the surface of the vascular graft. The wall of the vascular graft consists of two-layers of hydrogels:
(a) the inner portion of the wall of the vascular graft that is in direct contact with blood is made from non-bio-erodible (enzymatically stable) PVA hydrogel as described in the Examples 1 and 2 and
(b) the outer section of the wall of the vascular graft is actual scaffolding for tissue engineering made either from:
   (i) a non-bio-erodible PVA hydrogel (enzymatically stable) in a combination with bio-degradable biomaterial (enzymatically unstable) alginate although other biodegradable biomaterials such as poly(lactic acid) homo-polymer, poly(lactic acid-co-glycolic acid) copolymer, or poly(ethylene glycol) can be used or,
   (ii) electrospun polyvinyl alcohol fibers in a combination with bio-degradable biomaterial (enzymatically unstable) alginate although other biodegradable biomaterials such as poly(lactic acid) homo-polymer, poly(lactic-co-glycolic acid) copolymer, and polyethylene glycol can be used or,
   (iii) a bio erodible cellular or non-cellular PVA hydrogels.

When polyvinyl alcohol is used to make bio-erodible biomaterials one can use low molecular weights fully hydrolyzed or partially hydrolyzed PVA polymer. Molecular weights as low as 5,000 and as high as 50,000 can be used.

### Example 6. Vascular Grafts with Imbedded Sutures

This example deals with a method of imbedding sutures into walls at the both ends of vascular grafts.

There are a few reasons to justify importance of imbedding sutures into vascular grafts that need to undergo anastomosis to native blood vessels of any bore size. Imbedding suture is particularly important for anastomosis of small diameter vascular graft. All hydrogels have inherently low tear strength and thus will tear easily when sutured while using a needle and suture to puncture the wall of the hydrogel vascular graft. By imbedding sutures into each end of vascular grafts it would provide major benefits over classical suturing because imbedded sutures will provide tear-free suturing of hydrogels, minimizing blood leaks at the site of anastomosis and will allow fast suturing at the site of anastomosis. In using embedded sutures in the walls of vascular grafts, one does not need to puncture the hydrogel wall where one is likely to create sites for tear initiation and blood leaks.

One can certainly use classical suturing by puncturing the walls at the ends of vascular graft with a needle and suture and tying the suture to the wall of a blood vessel. There are multiple problems with the use of classical suturing of hydrogels. For instance, when the suture penetrates through the hydrogel it forms a hole, which becomes enlarged when the suture is under tension, and the suturing will become loose since hydrogels are elastomers. Also, such an enlarged hole in the hydrogel easily undergoes tears.

A more desirable method of anastomosis is to use sutures imbedded into the hydrogel walls, which would completely eliminate the drawbacks of classical suturing. The criterion for the selection of material from which sutures are made is that the material should form hydrogen bonds with PVA hydrogels. The ability of sutures forming hydrogen bonds with the PVA hydrogel matrix is critical since sutures must be cohesively bonded (not adhesively bonded) to the PVA hydrogel. This means that the suture will never be detached or pulled out of the hydrogel, or de-bonded, unless the hydrogel matrix undergoes tearing and the vascular graft undergoes full mechanical destruction.

Materials that can be used for making such sutures are PVA fibers, silk, cellulose-based fibers, or any other material such as surface-modified PE fibers that are capable of forming hydrogen bonds with the PVA hydrogel matrix. PVA fibers are preferred because of their intrinsic high strength is higher than any other known man-made fiber, especially if the PVA fibers are ultra-drawn.

Suture imbedding was done manually into the walls of vascular grafts at the stage prior to subjecting the vascular graft construction to low temperature induced hydrogel crosslinking. Robotic arms with precision placement of sutures into the wall of vascular graft is preferred.

### Example 7. Vascular Grafts Made from Composite PVA Hydrogels for Integration of the Permanent Vascular Graft into Surrounding Tissue

A vascular graft having a 3.5 mm inner diameter and 1.5 mm thick walls was made from a composite PVA Hydrogel. This composite hydrogel consisted of two PVA Hydrogel layers, that is, an inner bulk PVA Hydrogel layer and outer cellular PVA Hydrogel layer.

The inner PVA hydrogel layer is made from a bulk hydrogel that provides a smooth, enzymatically stable and non-thrombogenic surface for unimpeded long-term blood flow. This bulk hydrogel is made from PVA polymer having 150,000 molecular weight and >99.95+% of hydrolysis, which results in non-immunogenic and non-thrombogenic hydrogel surfaces based on procedures outlined in Example 1 and Example 3 and the outer layer is cellular PVA hydrogel made using pore-forming methods such as preselected sizes and shapes of salt particles that were then removed by washing out following the well-known technique of pore-forming process used industrially to make cellular materials.

The double layer vascular graft was formed by first submersing a rod having diameter of 3.5 mm into a PVA solution like the one prepared in Example 1 or Example 2. The surface of rod was wiped off leaving thin layer of PVA Hydrogel that will serve as the inner surface for blood contact. The rod was placed in a freezer at −18° C. for 30 min. without freezing. The rod was taken out of the freezer and submersed again into the PVA solution. Then, salt particles were thoroughly embedded by compacting them into the PVA solution and then placed into a tubular mold with a 7.5 mm inner diameter to arrive at the final outer diameter of composite vascular graft. The assembly was placed into a freezer at −18° C. for 8 hours. This allowed the hydrogel to physically crosslink. The salt particles were removed by washing out with water using a standard® procedure. In one example, the pores were loaded with bioactive glass having 4585 Bioglass® composition. Bioglass was supplied by NovaMin, Alachua, Florida, now part of GSK, a British pharmaceutical and biotechnology company. Bioglass is a synthetic hydroxyapatite that has high bioactivity in promoting tissue growth into cellular PVA structures.

### Example 8. Permanent Vascular Graft with Outer Surface Scaffolding for Tissue Integration

This example deals with methods of making a PVA Hydrogel vascular graft that is composed of two layers. The inner PVA hydrogel layer is made from a bulk hydrogel that provides a smooth, enzymatically stable and non-thrombogenic surface for unimpeded long-term blood flow. This bulk hydrogel is made from PVA polymer having 150,000 molecular weight and >99.5+% of hydrolysis, which results in non-immunogenic and non-thrombogenic hydrogel surfaces based on procedures outlined in Example 1 and Example 3.

The outer layer of the vascular graft is actually the scaffolding for tissue engineering made from different biomaterials that will guide and accelerate the integration of vascular grafts with the surrounding tissue. The outer scaffolding can be made of a variety of materials such as

electrospun PVA fibers or said fibers in a combination with bio-degradable biomaterial that is enzymatically unstable alginate although another biodegradable biomaterial such as poly(lactic acid) homo-polymer, poly(lactic-co-glycolic acid) copolymer, or polyethylene glycol, can be used or, silk or cellulose-based fibers can be used.

### Example 9. Vascular Grafts Made from Bulk PVA Hydrogels Reinforced with Braided Fiber

The fiber-reinforced tubes serving as the vascular grafts were made from a PVA polymer having a molecular weight of 150,000 and >99.9% of hydrolysis. The procedure for dissolution of polymer is described in Example 1.

Multi-filament braided silk-based fibers with a 100 μm diameter of single fibers was used for reinforcement of the PVA hydrogel-based vascular graft walls. Silk was used only for reinforcement of the walls of the vascular grafts and hence these fibers did not extend beyond the body of the tubular vascular graft. Silk, when imbedded into a PVA hydrogel, will cohesively bond to the PVA hydrogel using H-bonds. This means that surfaces of the silk fibers will never be exposed to the components of an immune system because once silk fibers have been coated with PVA Hydrogels they will always remain coated with hydrogel due to the strong propensity of silk to form H-bonds with the PVA hydrogel matrix. That means silk fibers will never activate an immune system and thus will never induce inflammatory responses such as a foreign body response.

Silk-based fiber reinforced PVA Hydrogel vascular grafts were made manually. The silk fibers were first wound around a mandrel in the form of a solid rod having diameter of 3.5 mm. Once the desired pattern of wound fibers is achieved, the mandrel, with reinforcing fibers, is submersed into the PVA solution. The mandrel containing the fibers and the PVA solution is inserted into a tube that has ID of 6 mm which serves as a mold for the vascular graft that will assure that the vascular graft will have an OD of 6 mm. The whole assembly is then placed into a freezer at a temperature of −18° C. for 8 hours. DMSO was extracted using a water bath as described in the Example 1.

### Example 10. Bio-Erodible Vascular Grafts Made from Enzymatically Unstable PVA Hydrogels for the Regeneration of Vasculature

The procedure used for preparation of the bio-erodible vascular graft was practically the same as described in Example 1 and Example 3 except that the chosen PVA polymer for this example does not result in PVA Hydrogel stability in the physiological environment.

In this example the rate of bio-erosion of the vascular graft is controlled by the selection of PVA polymer having a lower molecular weight than in Example 1 and Example 2. In this example, the PVA polymer has 10,000 molecular weight and 99.95% degree of hydrolysis.

All other steps for preparation of a bio-erodible vascular graft that include preparation of the PVA solution, molding of the tubes for initiation of crosslinking and removal of DMSO are the same as described in the Example 1.

### Example 11. Reinforced Composite Vascular Graft Made by 3-D Tissue Printing

PVA solutions were made using a DMSO/Water mixed solvent and 10% PVA as described in Example 1. However, in this case two different PVA solutions were made. Solution one was made from a PVA having degree of hydrolysis 99.95%+ and molecular weight of 150,000. Solution two was made from a PVA having degree of hydrolysis of 99.95% and a molecular weight of 10,000. Once the solution was made and cooled down to 40° C., this solution was loaded with epithelial cells. The solution was made to contain 6% loading of the epithelial cells.

These solutions were then loaded into two separate syringes. Solutions were dispensed manually to create desired pattern of permanent support coming from solution one and bio-erodible PVA hydrogel scaffolding layer contacting cells that play role of tissue engineering.

In this case, tissue regeneration occurs only on the surface of the vascular graft and will aid the incorporation of the vascular graft into the surrounding tissue. Hence, the vascular graft becomes an integral part of the surrounding tissue.

Thus, what is disclosed and claimed in the instant invention, in one embodiment, is physically crosslinked polyvinyl alcohol hydrogels prepared using mixed solvents. The hydrogels contain continuous, uninterrupted immobilized water. The hydrogels have at least 88% by weight water content or higher and a syndiotacticity of fifty percent or greater.

The hydrogels of this invention are unique. They must have a syndiotacticity of fifty percent or greater, have a molecular weight of at least 500 and up to 200,000, preferable, a molecular weight distribution of 2.5 or lower, a head-to-tail monomer orientation of 98 percent or higher, total branching of 1 percent or lower, total disordered stereotacticity of 70 percent or lower and a preferred degree of hydrolysis of 99.5 percent or greater.

What is claimed is:

1. A synthetic hemocompatible vascular graft manufactured from polyvinyl alcohol-based biomaterials, said polyvinyl alcohol-based biomaterials having a water content of at least 88 weight percent, a molecular weight equal to or greater than 500 g/mole, a molecular weight distribution of 2 to 6 units, a degree of hydrolysis of 99.5% or greater, a syndiotacticity of fifty percent or greater, a head-to-tail monomer orientation of 98 percent or higher, total branching of 1 percent or lower, total disordered stereotacticity of 70 percent or lower, and containing crystalline segments of polyvinyl alcohol.

2. A synthetic hemocompatible vascular graft as claimed in claim 1 wherein said graft is reinforced with fibers.

3. A synthetic hemocompatible vascular graft as claimed in claim 1 wherein the wall thickness of said grafts range from 200 to 1,500 μm.

4. A synthetic hemocompatible vascular graft as claimed in claim 1 wherein said graft has pre-embedded sutures cohesively attached thereto.

5. A process for manufacturing a synthetic hemocompatible vascular graft, said process comprising:
i) providing a polyvinyl alcohol polymer having a molecular weight equal to or greater than 500 g/mole;
ii) mixing said polyvinyl alcohol polymer with a mixed solvent in a vessel;
iii) heating said mixture of i) and ii) under a nitrogen blanket while stirring for at least 2 hours;
iv) forming said vascular graft;
v) cooling said vascular graft at less than 0° C. for at least 2 hours to form crystalline segments of polyvinyl alcohol material;
vi) allow said vascular graft to come to room temperature;
vii) extracting any remaining said solvent from said graft.

6. The process as claimed in claim 5 wherein said mixed solvent is water and dimethylsulfoxide.

7. The process as claimed in claim 5 wherein said mixed solvent is water and glycerin.

8. A bio-erodible synthetic hemocompatible vascular graft manufactured from polyvinyl alcohol-based biomaterials, said polyvinyl alcohol-based biomaterials having a water content of at least 88 weight percent, a molecular weight of 500 g/mole or greater, and containing crystalline segments of polyvinyl alcohol.

9. A synthetic hemocompatible vascular graft manufactured by the process as claimed in claim 5.

10. A bio-erodible synthetic hemocompatible vascular graft manufactured by the process as claimed in claim 5.

11. A synthetic hemocompatible vascular graft as claimed in claim 1 when coated, said coating selected from:
(i) an enzymatically stable non-bio-erodible polyvinyl alcohol hydrogel combined with enzymatically unstable bio-degradable biomaterial such as alginate, poly(lactic acid) homo-polymer, poly(lactic-co-glycolic acid) copolymer, and polyethylene glycol;
(ii) electrospun polyvinyl alcohol fibers combined with enzymatically unstable bio-degradable biomaterial such as alginate, poly(lactic acid) homo-polymer, poly(lactic-co-glycolic acid) copolymer, and polyethylene glycol;
(iii) a bio erodible cellular or non-cellular polyvinyl alcohol hydrogel.

12. A reinforced composite vascular graft as claimed in claim 1 when manufactured by 3-D tissue printing.
